(19) United States
(12) Patent Application Publication
(10) Pub. No.: US 2013/0260413 A1
(43) Pub. Date: Oct. 3, 2013

(54) ANALYSIS RESULT PROVIDING SYSTEM, SAMPLE ANALYZING SYSTEM, ANALYSIS RESULT PROVIDING APPARATUS, SAMPLE ANALYSIS RESULT PROVIDING METHOD, AND STORAGE MEDIUM

(71) Applicant: SYSMEX CORPORATION, Kobe-shi (JP)

(72) Inventors: Naoto KOSHIMURA, Kobe-shi (JP); Kyozo FUJITA, Akashi-shi (JP)

(73) Assignee: SYSMEX CORPORATION, Kobe-shi (JP)

(21) Appl. No.: 13/828,224

(22) Filed: Mar. 14, 2013

(30) Foreign Application Priority Data
Mar. 30, 2012 (JP) 2012-081896

(51) Int. Cl. G06F 19/26; G01N 33/50

(52) U.S. Cl. CPC G06F 19/26; G01N 33/50
USPC 435/34; 435/288.7; 436/69; 436/501; 422/68.1

(57) ABSTRACT

An analysis result providing system comprising, a sample analyzing section which analyzes a sample collected from a subject, a validation section which validates an analysis result obtained by the sample analyzing section, an e-mail sending section which sends, when an analysis result of the sample collected from the subject has been validated by the validation section, an e-mail for providing the analysis result; and a handheld device which is carried by the subject and which is capable of receiving the e-mail sent by the e-mail sending section and displaying a content of the e-mail.

### FIELD OF THE INVENTION

The present invention relates to an analysis result providing system for providing results of analysis of a sample such as blood or urine collected from a subject such as a patient, to the subject or the like, and relates to a sample analyzing system for analyzing samples, an analysis result providing apparatus, a sample analysis result providing method, and a storage medium.

### BACKGROUND

In hospitals, a sample such as blood or urine collected from a subject is tested by being analyzed by an automatic analyzer. The analysis result of the sample is provided to a doctor, and the doctor examines the subject based on the analysis result. The place where an automatic analyzer is installed, such as a laboratory, is often far from an examination room of a doctor, and thus, in a well-equipped hospital, an analysis result is provided to a doctor through communication via an in-hospital network installed in the hospital.

However, since construction of such an in-hospital network system requires huge costs, hospitals that do not have in-hospital network systems also exist. For example, in hospitals in some developing countries, an analysis result obtained by an automatic analyzer is printed on a sheet of paper in a laboratory, and the subject receives the sheet of paper and brings it to the doctor.

International Publication No. WO2007105198 discloses the following: specimen blood collected from a subject is dropped on a specimen collection strip; the specimen collection strip is inserted into a specimen collector; after the specimen is chemically prepared, a photograph of the specimen is taken; and the photograph of the specimen is subjected to image analysis, whereby an analysis result is obtained. In International Publication No. WO2007105198, the obtained analysis result is automatically transmitted to the subject and the medical authority.

When a failure, an abnormality, or the like has occurred in an automatic analyzer, analysis of a sample may not be normally performed, and even when an analysis result is obtained, the analysis result may not be accurate. However, in the method disclosed in International Publication No. WO2007105198 described above, the analysis result obtained by the specimen collector is transmitted to the subject and the medical authority regardless of whether the analysis result is accurate or not. Accordingly, a doctor cannot examine the subject appropriately based on such an analysis result.

The present invention has been made in view of the above situations. A main object of the present invention is to provide an analysis result providing system, a sample analyzing system, an analysis result providing apparatus, a sample analysis result providing method, and a storage medium that allow provision of an analysis result useful for examination of a subject performed by a doctor.

### SUMMARY OF THE INVENTION

The scope of the present invention is defined solely by the appended claims, and is not affected to any degree by the statements within this summary.

A first aspect of the present invention is an analysis result providing system comprising: a sample analyzing section which analyzes a sample collected from a subject; a validation section which validates an analysis result obtained by the sample analyzing section; an e-mail sending section which sends, when an analysis result of the sample collected from the subject has been validated by the validation section, an e-mail for providing the analysis result; and a handheld device which is carried by the subject and which is capable of receiving the e-mail sent by the e-mail sending section and displaying a content of the e-mail.

A second aspect of the present invention is a sample analyzing system comprising: a sample analyzing section which analyzes a sample collected from a subject; a validation section which validates an analysis result obtained by the sample analyzing section; and an e-mail sending section which sends, when an analysis result of the sample collected from the subject has been validated by the validation section, an e-mail for providing the analysis result to a handheld device carried by the subject.

A third aspect of the present invention is an analysis result providing apparatus, comprising: an analysis result obtaining section which obtains an analysis result of a sample collected from a subject; a validation section which validates an analysis result obtained by the analysis result obtaining section; and an e-mail sending section which sends, when an analysis result of the sample collected from the subject has been validated by the validation section, an e-mail for providing the analysis result to a handheld device carried by the subject.

A forth aspect of the present invention is a method of providing a sample analysis result, comprising: an analyzing step of analyzing a sample collected from a subject; a validation step of validating an analysis result obtained in the analyzing step; a sending step of sending, when an analysis result of the sample collected from the subject has been validated in the validation step, an e-mail for providing the analysis result to a handheld device carried by the subject; and a receiving step of the subject receiving the e-mail by means of the handheld device.

### BRIEF DESCRIPTION OF THE DRAWINGS

FIG. 1 is a schematic diagram showing a configuration of an analysis result providing system according to Embodiment 1;

FIG. 2 is a block diagram showing a configuration of a sample analyzer;

FIG. 3 is a block diagram showing a configuration of an information processing unit;

FIG. 4 is a block diagram showing a configuration of a test information management apparatus according to Embodiment 1;

FIG. 5 is a schematic diagram showing a configuration of a subject information database;

FIG. 6 is a block diagram showing a configuration of a mail server;

FIG. 7 is a flow chart showing the procedure of an analysis result notification process performed by the test information management apparatus according to Embodiment 1;

FIG. 8 is a schematic diagram showing a configuration of a not-validated item list;

FIG. 9 shows an example of the text of an analysis result notification e-mail;

FIG. 10 is a flow chart showing the procedure of a manual validation process performed by using a test information management apparatus;

FIG. 11 shows an example of an analysis result displaying screen;

FIG. 12 is a flow chart showing the procedure of a list check process performed by a test information management apparatus;

FIG. 13 shows an example of the text of an analysis result notification e-mail;

FIG. 14 shows an example of the text of an analysis result notification e-mail;

FIG. 15 is a flow chart showing the procedure of an invalid data deletion process performed by a test information management apparatus;

FIG. 16 shows the text of an e-mail for making notification that sending of analysis results has been completed;

FIG. 17 is a flow chart showing the procedure of a sample analysis process performed by a sample analyzer according to Embodiment 2;

FIG. 18 is a flow chart showing the procedure of an analysis result notification process performed by a test information management apparatus according to Embodiment 2;

FIG. 19 is a schematic diagram showing a configuration of an analysis result providing system according to Embodiment 3;

FIG. 20 is a block diagram showing a configuration of a web server;

FIG. 21 is a flow chart showing the procedure of an analysis result notification process performed by a test information management apparatus according to Embodiment 3; and

FIG. 22 is a schematic diagram showing a configuration of an analysis result providing system according to another embodiment.

### DETAILED DESCRIPTION OF THE PREFERRED EMBODIMENTS

The preferred embodiments of the present invention will be described hereinafter with reference to the drawings.

### Embodiment 1

### Configuration of Analysis Result Providing System

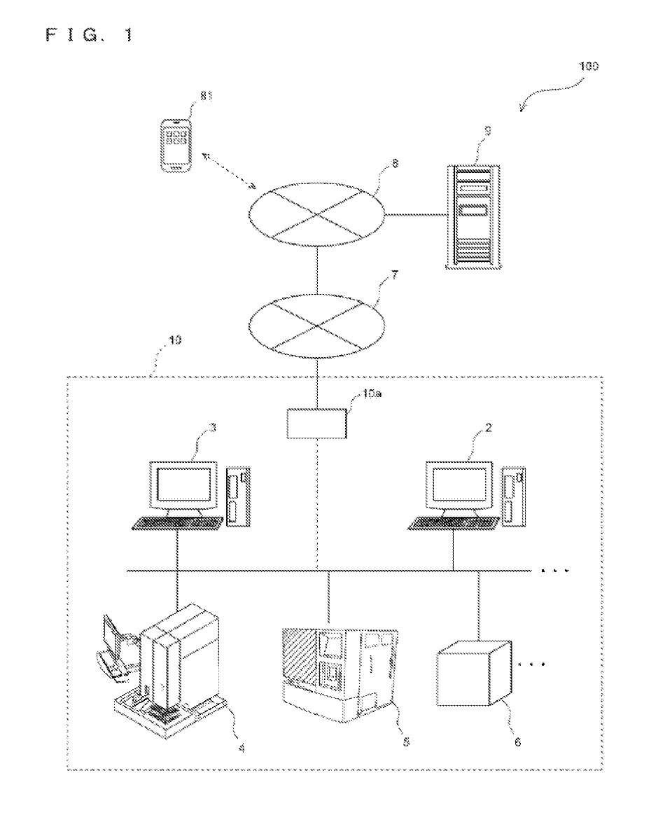

FIG. 1 is a schematic diagram showing a configuration of an analysis result providing system according to the present embodiment. An analysis result providing system 100 mainly includes a sample analyzing system 10, a mail server 9, and a handheld device 81.

The sample analyzing system 10 is installed in a medical facility such as a hospital, and includes a test information management apparatus 2, a subject information management apparatus 3, and sample analyzers 4, 5, and 6. The medical facility is provided with a LAN (local area network). The test information management apparatus 2, the subject information management apparatus 3, and the sample analyzers 4, 5, and 6 are each connected to the LAN, and can perform data communication with each other. The in-hospital LAN is connected to Internet 7 via a gateway 10a.

The mail server 9 is a mail server for mobile phones owned by a communications carrier. The mail server 9 is connected to a mobile phone network 8 owned by the communications carrier. The handheld device 81 carried by a subject can communicate with the mobile phone network 8 through wireless communication, and can communicate with another handheld device or the mail server 9 that can similarly use the mobile phone network 8. Further, the mobile phone network 8 and the Internet 7 are interconnected to each other. Accordingly, the test information management apparatus 2, and the sample analyzers 4, 5, and 6 can perform data communication with the mail server 9.

<Configuration of the Sample Analyzer 4>

The sample analyzer 4 is a multi-item blood cell analyzer. The sample analyzer 5 is a blood coagulation measuring apparatus, and the sample analyzer 6 is an immune analyzer. That is, the sample analyzers 4, 5, and 6 respectively perform different types of analyses.

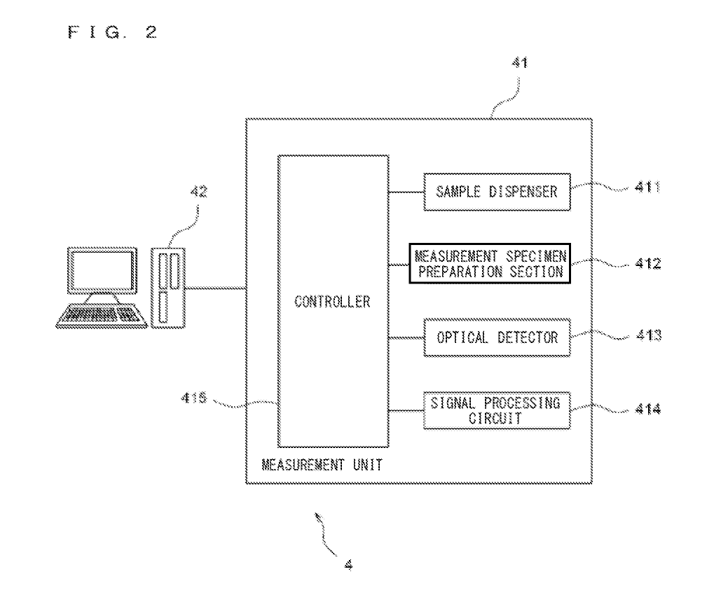

FIG. 2 is a block diagram showing a configuration of the sample analyzer 4. The sample analyzer 4 is a multi-item blood cell analyzer that uses optical flow cytometry. The sample analyzer 4 obtains scattered light intensity, fluorescence intensity, and the like regarding blood cells contained in a blood sample. Based on such obtained information, the sample analyzer 4 classifies the blood cells contained in the sample into their respective types, and counts blood cells of each type. The sample analyzer 4 generates and displays a scattergram in which the blood cells are classified and colored differently based on their respective types. The sample analyzer 4 includes a measurement unit 41 which measures blood samples, and an information processing unit 42 which processes measurement data outputted from the measurement unit 41 and displays analysis results of blood samples.

As shown in FIG. 2, the measurement unit 41 includes a sample dispenser 411, a measurement specimen preparation section 412, an optical detector 413, a signal processing circuit 414, and a controller 415.

The sample dispenser 411 includes an aspiration tube (not shown). The sample dispenser 411 pierces, with the aspiration tube, the cap of a sample container in a sample rack transported by a sample transporting apparatus, and then aspirates a blood sample from the sample container. The measurement specimen preparation section 412 includes a mixing container (not shown) and mixes and agitates the blood sample dispensed by the sample dispenser 411, a reagent, and a diluent together, to prepare a measurement specimen.

The optical detector 413 includes a flow cell (not shown). When the measurement specimen is supplied into the flow cell, a thin flow of the measurement specimen is formed. Then, the measurement specimen is irradiated; and a side scattered light signal, a forward scattered light signal, and a fluorescence signal are obtained through an optical sensor. These signals are outputted to the signal processing circuit 414. The signal processing circuit 414 is a circuit that processes electrical signals outputted from the optical detector 413. The signal processing circuit 414 obtains parameters such as peaks and pulse widths of the side scattered light signal, the forward scattered light signal, and the fluorescence signal.

The controller 415 includes a CPU and a memory. The controller 415 controls the sample dispenser 411, the measurement specimen preparation section 412, the optical detector 413, and the signal processing circuit 414, in accordance with analysis items provided from the information processing unit 42, and causes them to perform measuring operations corresponding to the analysis items. Further, the controller 415 is configured to transmit measurement data including the above parameters obtained by the signal processing circuit 414, to the information processing unit 42.

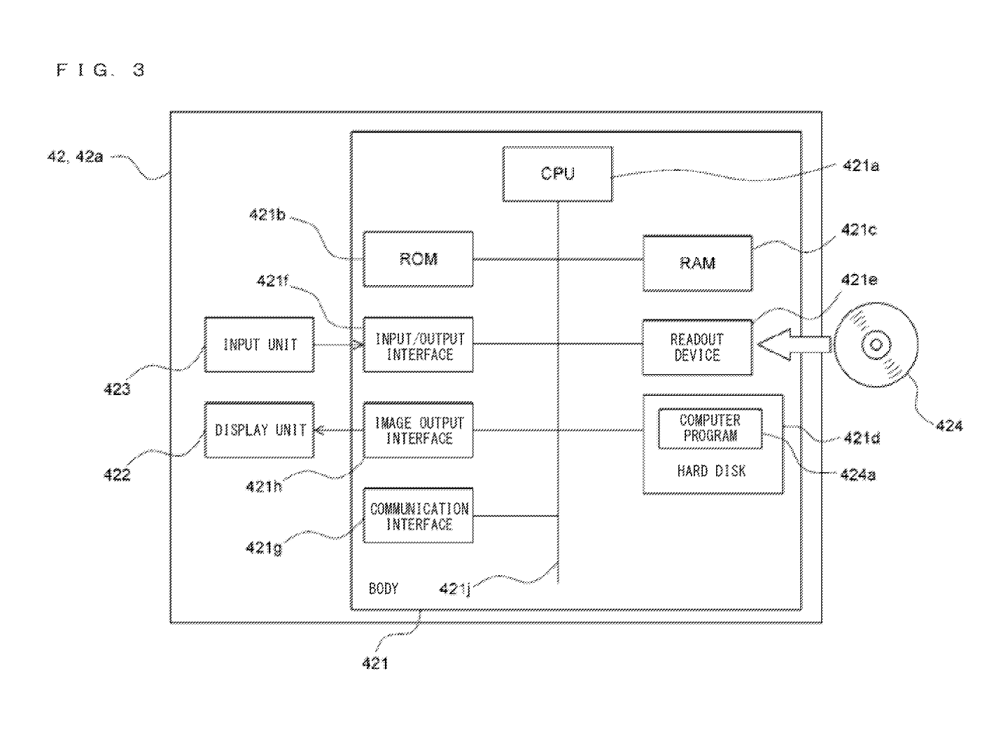

Next, a configuration of the information processing unit 42 will be described. The information processing unit 42 is composed of a computer. FIG. 3 is a block diagram showing the configuration of the information processing unit 42. The information processing unit 42 is realized by a computer 42a. As shown in FIG. 3, the computer 42a includes a body 421, a display unit 422, and an input unit 423. The body 421 includes a CPU 421a, a ROM 421b, a RAM 421c, a hard disk 421d, a readout device 421e, an input/output interface 421f, a communication interface 421g, and an image output interface 421h. The CPU 421a, the ROM 421b, the RAM 421c, the hard disk 421d, the readout device 421e, the input/output interface 421f, the communication interface 421g, and the image output interface 421h are connected to each other by a bus 421j.

The readout device 421e can read, from a portable storage medium 424, a computer program 424a for causing the computer to function as the information processing unit 42, and can install the computer program 424a in the hard disk 421d.

The computer program 424a for causing the computer to function as the information processing unit 42 is installed in the hard disk 421d. By the CPU 421a executing the computer program 424a, the computer 42a functions as the information processing unit 42.

The information processing unit 42 having the above configuration obtains order information for sample measurement, from the test information management apparatus 2. In the order information, a sample ID that specifies a sample to be analyzed and analysis items for which analyses are performed are designated.

When order information is obtained, the sample dispenser 411 aspirates a sample from a sample container, the measurement specimen preparation section 412 mixes the sample and a relevant reagent, whereby a measurement specimen for each analysis item designated in the order information is prepared. When the measurement specimens have been prepared, the optical detector 413 measures the sample for each analysis item, and measurement data is outputted from the signal processing circuit 414. The measurement data is provided to the information processing unit 42, and the CPU 421a of the information processing unit 42 performs analysis processing, whereby analysis results are obtained. The obtained analysis results are displayed on the display unit 422, and also transmitted to the test information management apparatus 2.

Although detailed description of a configuration of the sample analyzer 5 is omitted, in the sample analyzer 5, a blood sample is dispensed from a sample container into a cuvette for optical measurement, for each analysis item designated in order information provided from the test information management apparatus 2. While the cuvette is heated at a predetermined temperature, a reagent is dispensed into the cuvette, and absorbance and the like of the specimen in the cuvette is measured. In this manner, blood coagulation measurement is performed. Further, detailed description of a configuration of the sample analyzer 6 is also omitted. In the sample analyzer 6, a blood sample is dispensed from a sample container into a cuvette for optical measurement, for each analysis item designated in order information provided from the test information management apparatus 2. Also, a capture antibody (R1 reagent) and magnetic particles (R2 reagent) are dispensed into the cuvette. Thus, the capture antibody (R1 reagent) bound to the antigen contained in the sample is caused to be bound to the magnetic particles (R2 reagent). Then, the antigen, the capture antibody, and the magnetic particles, which are bound, are attracted to a magnet for primary BF (bound free) separation. Accordingly, the R1 reagent containing the capture antibody that is unreacted (i.e., free) is removed. Further, in the sample analyzer 6, a labeled antibody (R3 reagent) is dispensed into the cuvette. Then, the antigen bound to the magnetic particles is caused to be bound to the labeled antibody (R3 reagent). Then, the magnetic particles, the antigen, and the labeled antibody, which are bound, are caused to be attracted to a magnet for secondary BF separation. Accordingly, the R3 reagent containing the labeled antibody that is unreacted (i.e., free) is removed. Then, after a dispersion liquid (R4 reagent) and a luminescent substrate (R5 reagent) that emits light during a reaction with the labeled antibody are added in the cuvette, the amount of light generated in the reaction between the labeled antibody and the luminescent substrate is measured. Through these steps, the sample analyzer 6 quantitatively measures the antigen contained in the sample, which is bound to the labeled antibody.

When having obtained analysis results, each of the sample analyzers 5 and 6 transmits the analysis results to the test information management apparatus 2.

<Configuration of the Test Information Management Apparatus 2>

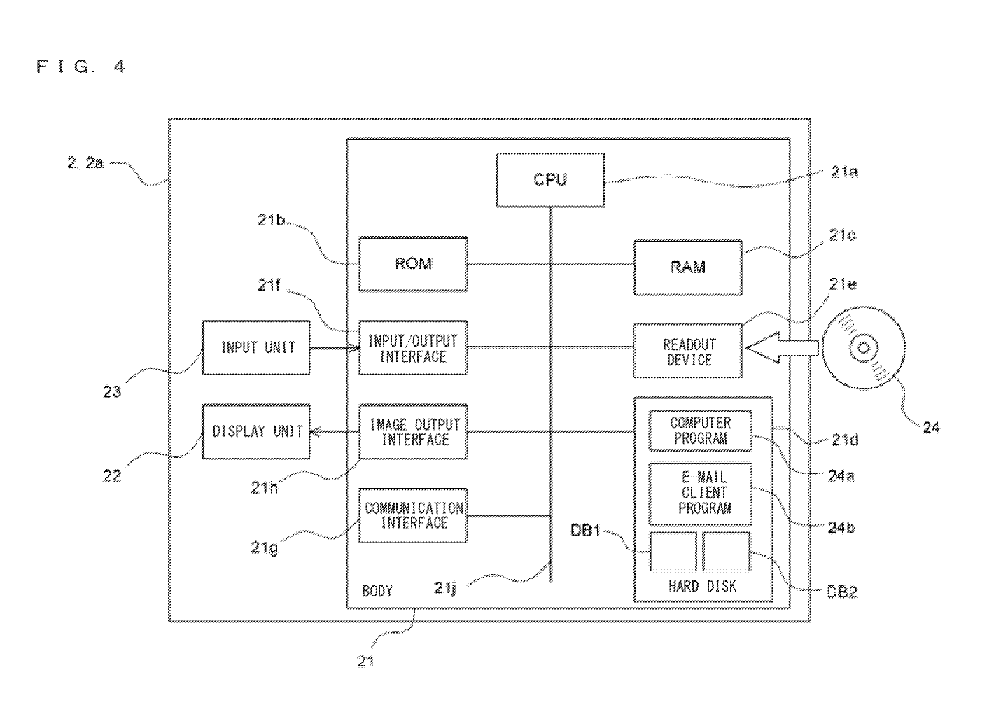

Next, a configuration of the test information management apparatus 2 will be described. FIG. 4 is a block diagram showing the configuration of the test information management apparatus 2 according to the present embodiment. The test information management apparatus 2 is realized by a computer 2a. As shown in FIG. 4, the computer 2a includes a body 21, a display unit 22, and an input unit 23. The body 21 includes a CPU 21a, a ROM 21b, a RAM 21c, a hard disk 21d, a readout device 21e, an input/output interface 21f, a communication interface 21g, and an image output interface 21h. The CPU 21a, the ROM 21b, the RAM 21c, the hard disk 21d, the readout device 21e, the input/output interface 21f, the communication interface 21g, and the image output interface 21h are connected to each other by a bus 21j.

The readout device 21e can read, from a portable storage medium 24, a computer program 24a for causing the computer to function as the test information management apparatus 2, and can install the computer program 24a in the hard disk 21d.

The computer program 24a for causing the computer to function as the test information management apparatus 2 is installed in the hard disk 21d. By the CPU 21a executing the computer program 24a, the computer 2a functions as the test information management apparatus 2.

Further, an e-mail client program 24b is installed in the hard disk 21d. By the CPU 21a executing the e-mail client program 24b, the computer 2a functions as a client of the e-mail system, and becomes able to send/receive e-mails. More specifically, a mail address of the medical facility (hereinafter referred to as “analysis result notification mail address”) is registered in the e-mail client program 24b. Accordingly, the test information management apparatus 2 can receive an e-mail addressed to the analysis result notification mail address, and can generate an e-mail using the analysis result notification mail address as a sender to send the e-mail.

Further, the hard disk 21d is provided with an order information database DB1 in which order information is registered, and an analysis result database DB2 for storing analysis results.

<Configuration of the Subject Information Management Apparatus 3>

Next, a configuration of the subject information management apparatus 3 will be described. The subject information management apparatus 3 is realized by a computer. Since the configuration of the computer is substantially the same as that of the test information management apparatus described above, the description thereof is omitted.

The hard disk of the subject information management apparatus 3 is provided with a subject information database DB3. FIG. 5 is a schematic diagram showing the configuration of the subject information database DB3. Information of subjects who attend or are hospitalized in the medical facility is stored in the subject information database DB3. The subject information database DB3 includes a field for storing a subject ID and a field for storing a mail address of a handheld device of the subject. Here, the mail address of a handheld device of a subject is a mail address which allows the subject to receive a mail by means of the handheld device. Such a mail address may be not only a mail address for the handheld device issued by the communications carrier, that is, a mail address which allows only that handheld device to send/receive an e-mail, but also any mail address, issued by an Internet service provider or the like, that allows sending/receiving an e-mail by means of an information terminal (for example, personal computer) other than the handheld device, as long as that mail address allows the handheld device to send/receive an e-mail.

<Configuration of the Mail Server 9>

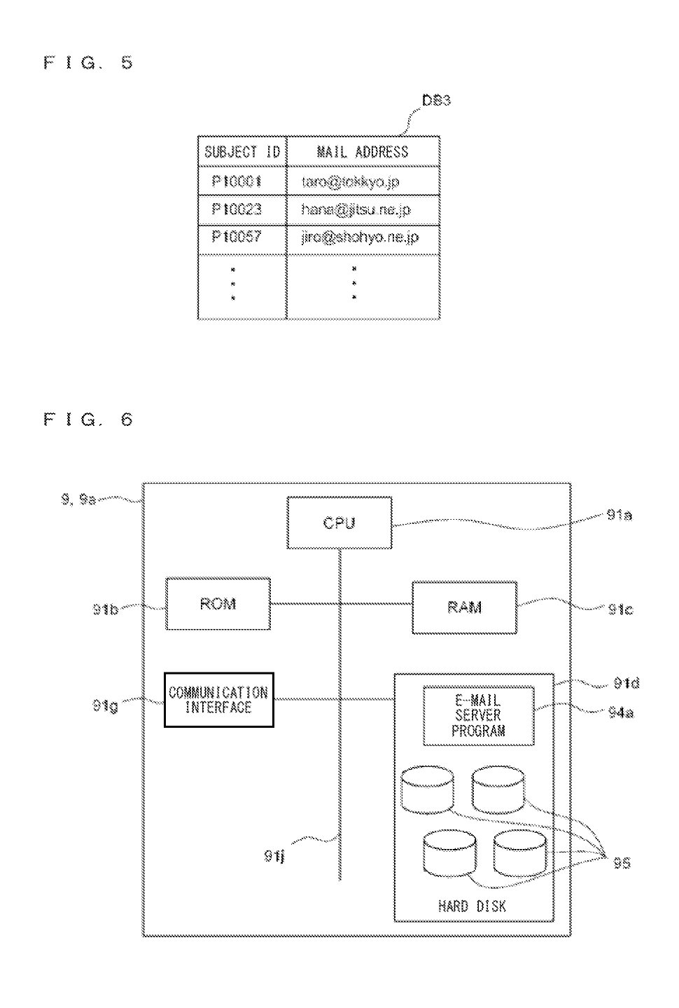

Next, a configuration of the mail server 9 will be described. FIG. 6 is a block diagram showing the configuration of the mail server 9. The mail server 9 is realized by a computer 9a. As shown in FIG. 6, the computer 9a includes a CPU 91a, a ROM 91b, a RAM 91c, a hard disk 91d, and a communication interface 91g. The CPU 91a, the ROM 91b, the RAM 91c, the hard disk 91d, and the communication interface 91g are connected to each other by a bus 91j.

An e-mail server program 94a for causing the computer to functions as the mail server 9 is installed in the hard disk 91d. By the CPU 91a executing the e-mail server program 94a, the computer 9a functions as the mail server 9.

Further, the hard disk 91d is provided with a mailbox 95 for each handheld device user. Each mailbox 95 is individually associated with a mail address, and in one mailbox 95, e-mails for the mail address associated with the mailbox 95 (that is, e-mails addressed to the handheld device of the user that corresponds to the mailbox 95) are stored.

Further, the mail server 9 can send an e-mail sent from a handheld device, to a mail address to which the e-mail has been addressed. For example, the mail server 9 can receive, from the handheld device 81, an e-mail addressed to the analysis result notification mail address sent by the handheld device 81, and can send the e-mail to the analysis result notification mail address. Accordingly, the e-mail is received by the test information management apparatus 2.

<Configuration of the Handheld Device 81>

Next, a configuration of the handheld device 81 will be described. The handheld device 81 has a function as an e-mail client. That is, the handheld device 81 is communicable with the mail server 9 via the mobile phone network 8, and can receive an e-mail stored in the mailbox for the handheld device 81 provided in the hard disk 91d of the mail server 9. Further, the handheld device 81 is provided with a display section and can display the content of the received e-mail.

The handheld device 81 can also send an e-mail. A subject can create an e-mail by operating the handheld device 81, and can send the created e-mail. The e-mail sent from the handheld device 81 is sent to its destination mail address via the mail server 9. It should be noted that in the present embodiment, the handheld device 81 may be a handheld device personally owned by the subject, or a handheld device lent or otherwise provided to the subject from the medical facility, the communications carrier, another organization, or an individual. That is, it is sufficient that the handheld device 81 is carried by the subject, and the handheld device 81 may or may not be a belonging to the subject. Further, in the case where the medical facility or the like lends a handheld device to a subject, the handheld device may be the one that is solely used for notification of analysis results.

[Operation of Analysis Result Providing System]

Next, operations performed by the analysis result providing system 100 according to the present embodiment will be described.

A subject visits a medical facility to be examined by a doctor, and has his or her blood collected prior to the examination. Order information of sample analysis for the subject is registered in the order information database DB1 of the test information management apparatus 2. The blood sample collected from the subject is transported to a laboratory where the sample analyzers 4, 5, and 6 are installed, and is subjected to analysis by the relevant sample analyzer 4, 5, or 6.

Prior to the analysis of the sample to be performed by the sample analyzer 4, 5, or 6, order information of the subject is transmitted from the test information management apparatus 2 to the sample analyzer 4, 5, or 6. The sample analyzer 4, 5, or 6 receives the order information, and performs sample analyses for analysis items designated in the order information. When analysis results have been obtained, the sample analyzer 4, 5, or 6 transmits the analysis results to the test information management apparatus 2. The test information management apparatus 2 receives the analysis results and registers them in the analysis result database DB2.

When blood of the subject is collected, the subject is notified of the analysis result notification mail address. Further, the subject is informed that when the subject sends an e-mail from the handheld device 81 to the analysis result notification mail address, the subject can be provided with his or her analysis results, and that the subject is supposed to be provided with the analysis results prior to the examination by the doctor, and to present the analysis results to the doctor.

The subject waits until the time for the examination by the doctor, and sends an e-mail from the handheld device 81 to the analysis result notification mail address during that time period, thereby requesting provision of the analysis results. The e-mail is sent to the analysis result notification mail address by the mail server 9.

<Analysis Result Notification Process>

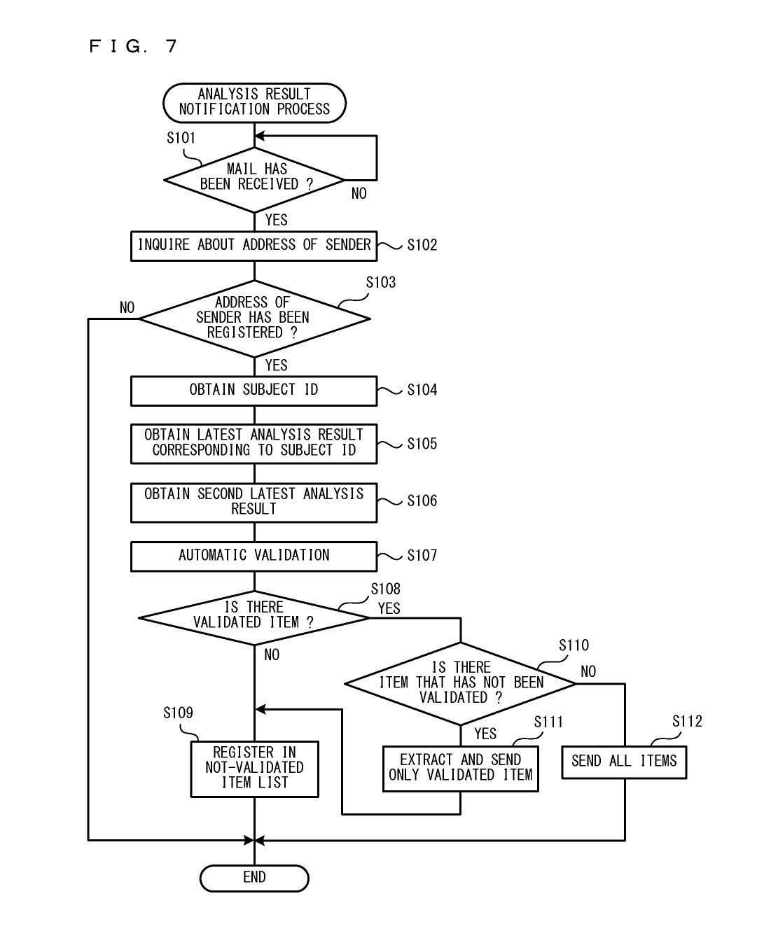

When the test information management apparatus 2 receives the e-mail sent from the handheld device 81 of the subject and addressed to the analysis result notification mail address (hereinafter, referred to as “analysis result notification request mail”), an analysis result notification process described below is executed by the CPU 21a of the test information management apparatus 2. FIG. 7 is a flow chart showing the procedure of the analysis result notification process performed by the test information management apparatus 2 according to the present embodiment. The CPU 21a of the test information management apparatus 2 determines whether an analysis result notification request mail sent from the handheld device 81 has been received (step S101). When the analysis result notification request mail has not been received (NO in step S101), the CPU 21a returns the process to step S101, and repeats confirmation of whether the analysis result notification request mail has been received. In this manner, the test information management apparatus 2 waits until receiving the analysis result notification request mail.

When the test information management apparatus 2 has received the analysis result notification request mail (YES in step S101), the CPU 21a requests the subject information management apparatus 3 to inquire about the mail address of the sender of the analysis result notification request mail (step S102). The subject information management apparatus 3 determines whether the mail address of the sender, that is, the mail address of the handheld device of the subject, has been registered in the subject information database DB3, thereby inquiring about the mail address of the sender. The inquiry result about the mail address of the sender is transmitted from the subject information management apparatus 3 to the test information management apparatus 2. In the case where the mail address of the sender has been registered in the subject information database DB3, the data of the inquiry result about the mail address of the sender contains a subject ID corresponding to the mail address of the sender.

Upon receiving the inquiry result about the mail address of the sender, the CPU 21a of the test information management apparatus 2 determines whether the mail address of the sender has been registered in the subject information database DB3, based on the received inquiry result (step S103). When the mail address of the sender has not been registered in the subject information database DB3 (NO in step S103), the CPU 21a directly ends the process.

On the other hand, when the mail address of the sender has been registered in the subject information database DB3 (YES in step S103), the CPU 21a obtains the subject ID from the data of the inquiry result (step S104).

Next, the CPU 21a obtains latest analysis results of the subject from the analysis result database DB2, by using the obtained subject ID as a key (step S105). At this time, when latest analysis results have not been registered in the analysis result database DB2 yet, the CPU 21a waits until analysis results are transmitted from the sample analyzer 4, 5, or 6 and registered in the analysis result database DB2.

Upon obtaining the latest analysis results of the subject, the CPU 21a obtains second latest analysis results of the subject from the database DB2 (step S106).

Next, the CPU 21a performs an automatic validation process for the latest analysis results (step S107). In this process, it is determined, for each analysis item, whether a corresponding analysis result satisfies a predetermined reliability condition. An analysis result for an item that has satisfied the reliability condition is validated, and a validation flag, which is information indicating whether the analysis result has been validated, is set to ON for that analysis result. On the other hand, an analysis result for an item that has not satisfied the reliability condition is not validated, and the validation flag is set to OFF for that analysis result.

The CPU 21a determines whether there is at least one item that has been validated (step S108). When there is no item that has been validated (NO in step S108), the CPU 21a does not permit provision of the analysis results to the subject, registers all the items in a not-validated item list (step S109), and ends the process.

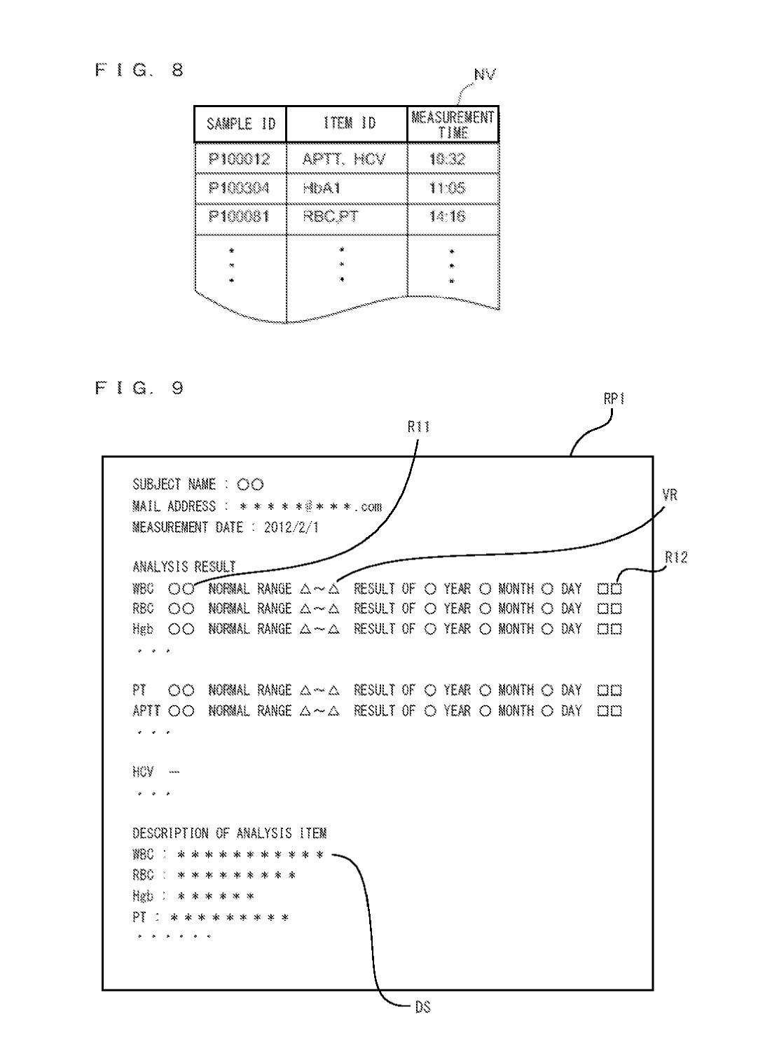

Here, the not-validated item list will be described. FIG. 8 is a schematic diagram showing a configuration of the not-validated item list. A not-validated item list NV is provided in the hard disk 21d of the test information management apparatus 2. The not-validated item list NV is a table including a field for registering a subject ID, a field for registering an item ID(s) for identifying an analysis item(s), and a field for registering the sample measurement time of its corresponding analysis results. In the field for registering a subject ID, the subject ID obtained in step S104 is registered. In the field for registering an item ID(s), each analysis item that was not validated in the automatic validation process, that is, the item ID of each analysis item for which the validation flag is set to OFF, is registered. Further, in the field for registering the measurement time, the measurement time for the latest analysis results obtained in step S105 is registered. Therefore, in the above case, all the analysis items included in the analysis result information are registered in the not-validated item list NV along with the subject ID and the measurement time.

In step S108, when there is at least one item that has been validated (YES in step S108), the CPU 21a determines whether there is at least one item that has not been validated (step S110). When there is at least one item that has not been validated (YES in step S110), the CPU 21a permits provision, to the subject, of the analysis result for each item that has been validated; and does not permit provision, to the subject, of the analysis result for each item that has not been validated. In this case, the CPU 21a extracts, from all of the latest analysis results, only the analysis result for each validated item, creates an e-mail addressed to the mail address of the handheld device of the subject (that is, the mail address of the sender), and sends the e-mail (step S111). In this process, an e-mail is created in which the analysis result for each validated item is described in the text. Further, the second latest analysis result for each validated item (hereinafter, referred to as “second latest value”) is also added to the text of the e-mail.

After performing the process of step S111, the CPU 21a registers each item that has not been validated in the not-validated item list (step S109), and ends the process. In this case, among the analysis items included in the analysis result information, only the analysis item(s) that has not been validated is registered in the not-validated item list NV, along with the subject ID and the measurement time. For example, in the case where analysis items “APTT” and “HCV” have not been validated, the item IDs, i.e., “APTT” and “HCV” are registered in the not-validated item list NV.

In step S110, when there is no item that has not been validated, that is, when all the analysis items have been validated (NO in step S110), the CPU 21a permits provision of the analysis results for all the analysis items to the subject. In this case, the CPU 21a creates an e-mail addressed to the mail address of the handheld device of the subject, in which all the latest analysis results are added to the text thereof, sends the e-mail (step S112), and ends the process. In this process, since all the analysis items have been validated, an e-mail is created in which the analysis results for all the analysis items are described in the text thereof. Moreover, the second latest values for all the analysis items are also added to the text of the e-mail.

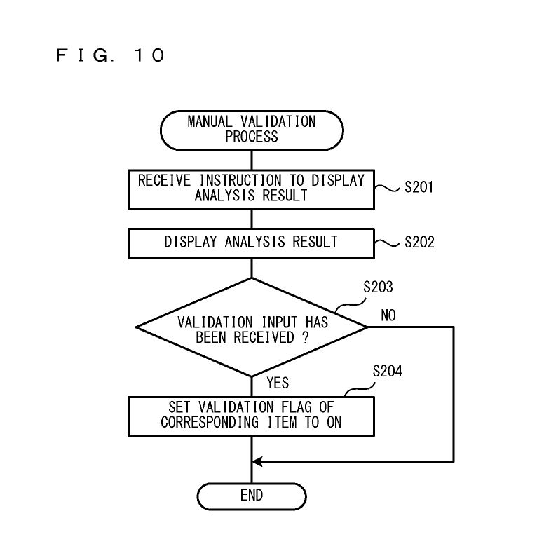

FIG. 9 shows the text of an analysis result notification e-mail. The subject name, the mail address of the handheld device of the subject, and the measurement date are indicated as character information in text RP1 of the analysis result notification e-mail. In addition, a latest analysis result R11 is shown for each analysis item. The analysis results R11 contained in the e-mail are only those that were validated in the automatic validation process. Further, for each analysis item, a normal range VR for an analysis result is shown to the right of its corresponding analysis result R11. The normal range VR is supplementary information for the subject. By confirming the normal range VR, the subject can know whether his or her analysis result is normal or abnormal, whether the analysis result is near the border of the normal range even when the analysis result is normal, or how far the analysis result is from the normal range when the analysis result is abnormal. To the right of the normal range VR, the date of the second latest measurement and a second latest value R12 are shown, for each analysis item.

Further, the text RP1 of the e-mail includes description DS of each analysis item. The description is supplementary information for the subject. By reading the description DS, the subject can know what the analysis item means.

Such an e-mail described above is sent from the test information management apparatus 2, and then is stored in the mailbox 95 of the mail server 9. The e-mail stored in the mailbox 95 is sent to the handheld device 81 of the subject, and the content of the e-mail is displayed on the display section of the handheld device 81. Accordingly, the subject is provided with the analysis results.

<Manual Validation Process>

When the analysis result notification process described above has been performed, and a part or all of the analysis results have failed to be validated, the corresponding item IDs that have not been validated are stored in the not-validated item list. Through a manual validation process below, an operator can manually validate the part or all of the analysis results that have failed to be validated.

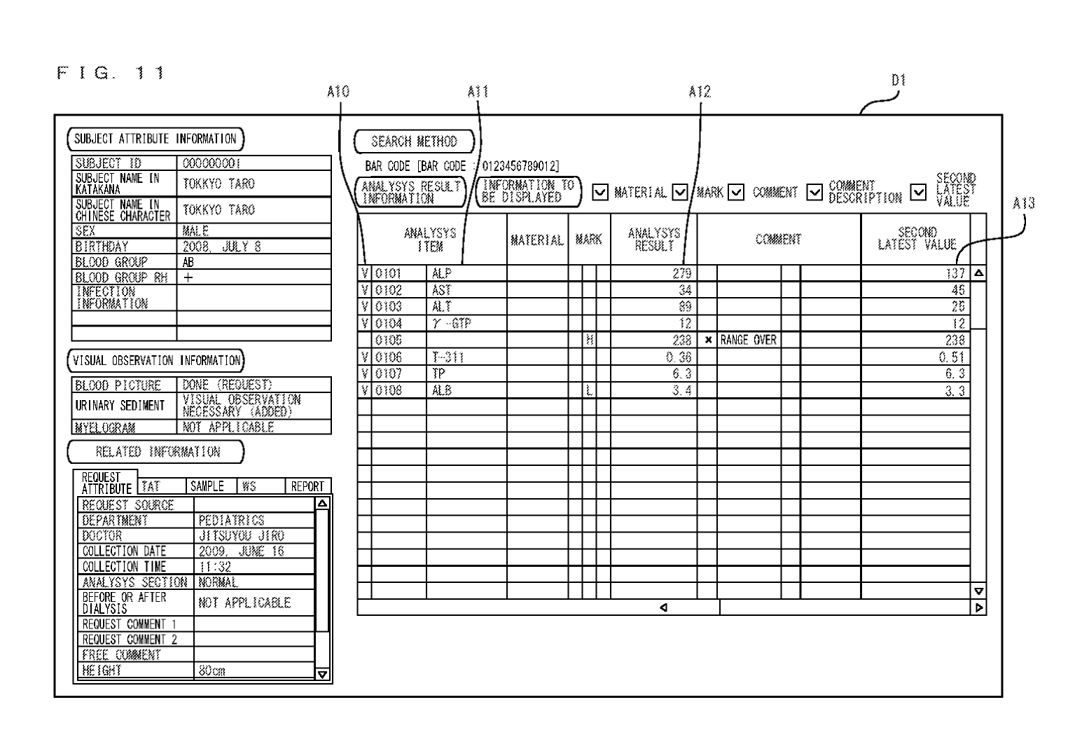

FIG. 10 is a flow chart showing the procedure of the manual validation process performed by using the test information management apparatus 2. By operating the input unit 23 of the test information management apparatus 2, the operator can instruct the test information management apparatus 2 to display analysis results. Upon receiving the instruction to display the analysis results (step S201), the CPU 21a causes the display unit 22 to display the analysis results (step S202).

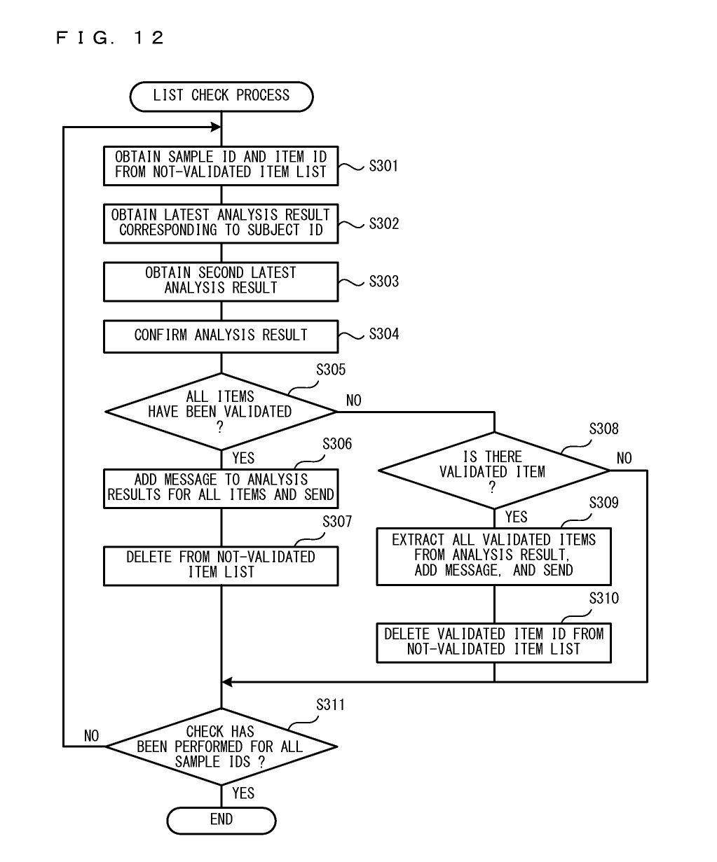

FIG. 11 shows an example of an analysis result displaying screen. On an analysis result displaying screen D1, analysis results of the subject are listed for the respective items. The analysis result displaying screen D1 includes an area A11 for displaying analysis items, an area A12 for displaying numerical value data of the analysis results, and an area A13 for displaying second latest values. The operator performs searching or the like of the analysis result database DB2 for target analysis results, by using the subject ID, the sample ID, or the like as a search key, thereby causing the analysis results of the subject to be displayed on the analysis result displaying screen D1. The analysis results of the subject are displayed for the respective analysis items in the area A12. At this time, second latest values of the subject are read from the analysis result database DB2, and are displayed for the respective analysis items in the area A13. The analysis results of the present analysis (present values) and the second latest values are displayed in a table such that one line corresponds to one analysis item. Further, the analysis result displaying screen D1 includes an area A10 for displaying, for each analysis item, information indicating that its corresponding analysis result has been validated (hereinafter, referred to as “validation information”). With respect to an analysis item for which the validation flag is set to ON in its analysis result, a symbol “V” representing the validation information is displayed in the area A10. With respect to an analysis item for which the validation flag is set to OFF, the validation information is not displayed in the area A10. Accordingly, the operator can understand, for each analysis item, whether the analysis result is validated.

Each cell of the area A10 can be selected by the operator clicking the mouse. When a cell in which the validation information is not displayed is selected, the validation information is displayed in the cell, and the validation flag for the corresponding analysis item is set to ON in the analysis result. In this manner, the operator can manually validate the analysis result.

The CPU 21a determines whether one or more validation inputs from the operator as described above have been received (step S203). When one or more validation inputs have been received (YES in step S203), the CPU 21a sets the validation flag(s) for the corresponding analysis item(s) to ON (step S204), and ends the manual validation process. On the other hand, when no validation input from the operator has been received (NO in step S203), the CPU 21a directly ends the manual validation process.

<List Check Process>

The test information management apparatus 2 periodically performs a list check process described below, to check whether there is an analysis result that has been newly validated through the manual validation process.

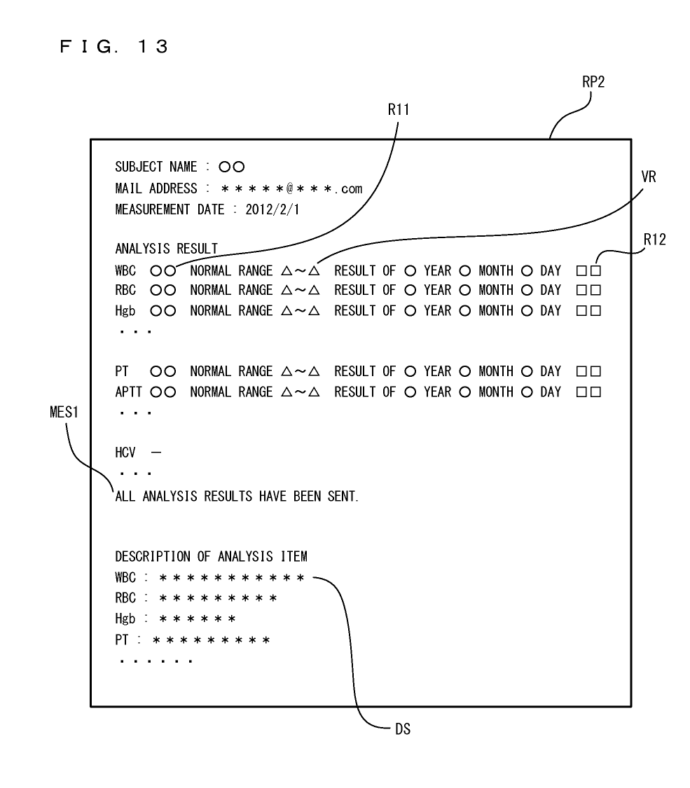

FIG. 12 is a flow chart showing the procedure of the list check process performed by the test information management apparatus 2. In the list check process, the CPU 21a first obtains one sample ID and item IDs corresponding to this sample ID from the not-validated item list NV (step S301). Then, using the obtained sample ID as a key, the CPU 21a obtains analysis results (the latest analysis results) from the analysis result database DB2 (step S302), and further obtains the second latest analysis results of the subject from the database DB2 (step S303).

The CPU 21a confirms, in the analysis results obtained in step S302, the validation flags for the analysis items corresponding to the item IDs obtained in step S301 (step S304). Next, the CPU 21a determines whether all of the analysis items registered in the not-validated item list NV have been validated regarding the analysis results (step S305). When all of the analysis items registered in the not-validated item list NV have been validated, that is, when there are no analysis items that have not been validated regarding the analysis results (YES in step S305), the CPU 21a permits provision of the analysis results to the subject, for all the items registered in the not-validated item list NV. In this case, the CPU 21a creates an e-mail in which all of the latest analysis results are added to the text, and sends the e-mail to the mail address of the handheld device of the subject corresponding to the sample ID (step S306).

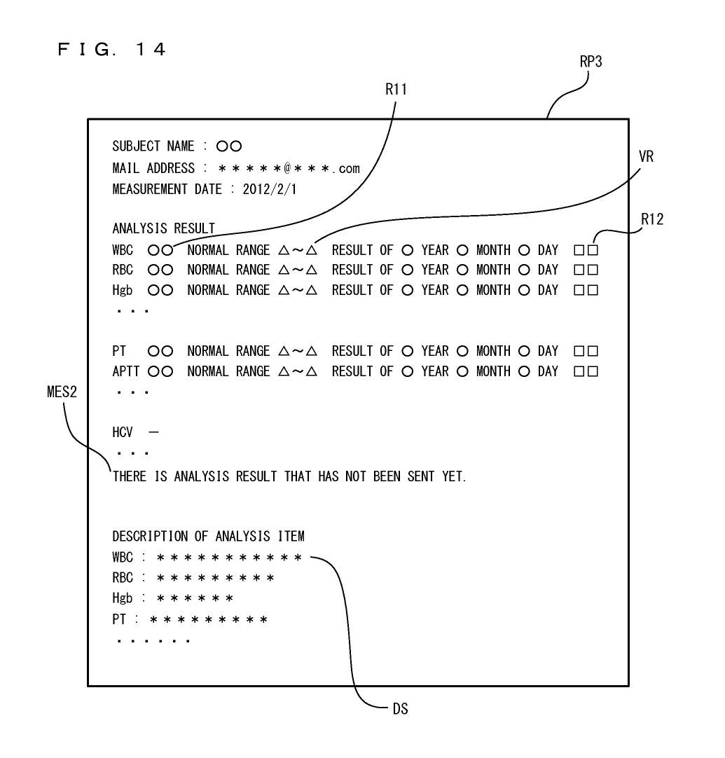

FIG. 13 shows the text of an analysis result notification e-mail sent in step S306. In the process of step S306, since all of the analysis items have been validated, an e-mail is created in which the analysis results for all of the analysis items are described in the text. That is, in mail text RP2, with respect to the sample of the subject, the analysis results R11 for all of the analysis items analyzed by the sample analyzer 4, 5, or 6 are listed. Further, also in this mail text RP2, the normal range VR for its corresponding analysis result and a corresponding second latest value R12 are shown for each analysis item, and the description DS for each analysis item is shown.

Further, the mail text RP2 includes a message MES1 expressed in character information “all analysis results have been sent.” The message MES1 allows the subject or the doctor to know that this is the last analysis result notification e-mail and that no more analysis result notification e-mail will be sent after this e-mail. Accordingly, the doctor can know that he or she may examine the subject, using the analysis results provided by this e-mail. Further, this e-mail provides the subject with the analysis results for all of the analysis items. Accordingly, the subject or the doctor can confirm the analysis results for all of the analysis items, simply by referring to this single e-mail.

When the analysis result notification e-mail as described above has been sent, the CPU 21a deletes data corresponding to the sample ID (sample ID and all of the item IDs) from the not-validated item list NV (step S307), and advances the process to step S311.

On the other hand, when there is at least one analysis item that has not been validated among the analysis items registered in the not-validated item list NV (NO in step S305), the CPU 21a determines whether there is at least one analysis item that has been validated among the analysis items registered in the not-validated item list NV (step S308). When there is at least one analysis item that has been validated among the analysis items registered in the not-validated item list NV (YES in step S308), the CPU 21a permits provision, to the subject, of the analysis result of each item that has been validated, among the items registered in the not-validated item list NV, and does not permit provision, to the subject, of the analysis result of each item that has not been validated. At this time, the CPU 21a extracts, from all of the latest analysis results, only the analysis result of each item that has been validated, creates an e-mail addressed to the mail address of the handheld device of the subject corresponding to the sample ID, and sends the e-mail (step S309).

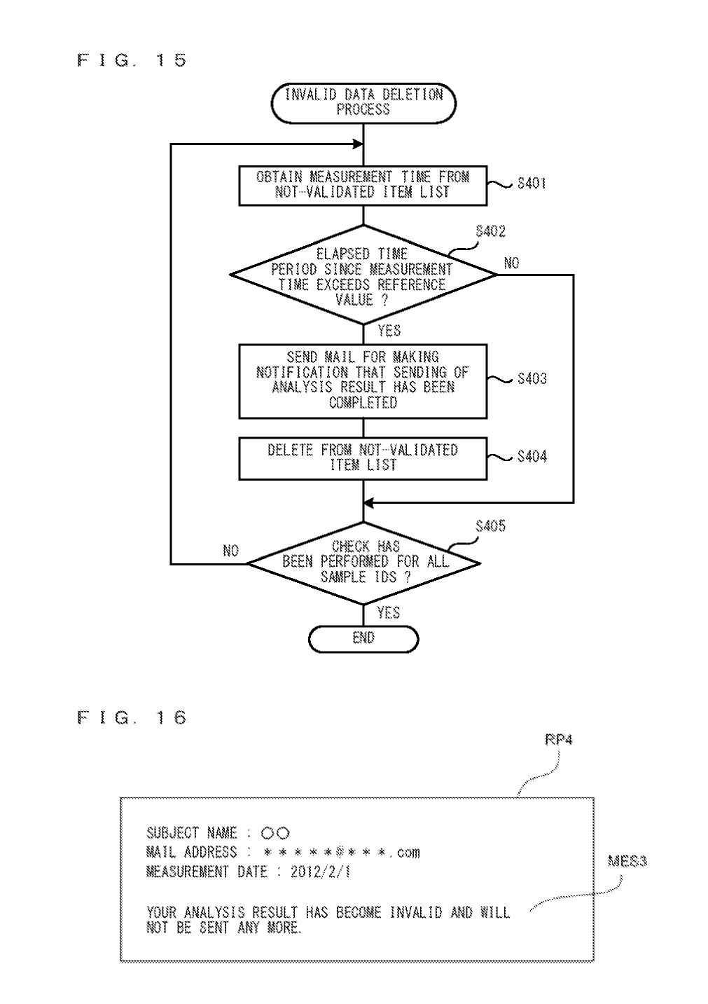

FIG. 14 shows the text of an analysis result notification e-mail sent in step S309. In the process of step S309, since a part of the analysis items have been validated, an e-mail is created in which analysis results for the validated analysis items are described in the text. That is, in mail text RP3, among the latest analysis results of the sample of the subject, analysis results R11 for the analysis items for which the validation flags are respectively set to ON are listed. Further, also in the mail text RP3, the normal range VR for its corresponding analysis result and a corresponding second latest value R12 are shown for each analysis item, and the description DS for each analysis item is shown.

Further, the mail text RP3 includes a message MES2 expressed in character information “there is an analysis result that has not been sent yet.” The message MES2 allows the subject or the doctor to know that there is a possibility that an analysis result notification e-mail will be sent after this e-mail. Accordingly, the doctor can know that, by using not only the analysis results provided by this e-mail, but also an additional analysis result if it is provided by another e-mail later, he or she can perform more appropriate examination. Further, the e-mail provides the subject with not only the analysis items registered in the not-validated item list NV but also the analysis results for all of the analysis items that have been validated. Accordingly, the subject or the doctor can confirm the analysis results for all of the analysis items that have been validated at that time point, simply by referring to this single e-mail.

When the analysis result notification e-mail as described above has been sent, the CPU 21a deletes the item IDs corresponding to the sample ID that have been validated from the not-validated item list NV (step S307), and advances the process to step S311.

On the other hand, when none of the analysis items registered in the not-validated item list NV have been validated yet (NO in step S308), the CPU 21a permits provision of the analysis results to the subject for none of the items registered in the not-validated item list NV. In this case, the CPU 21a advances the process directly to step S311.

In step S311, the CPU 21a determines whether the above-described check has been performed for all of the sample IDs registered in the not-validated item list NV (step S311). When there is a sample ID for which the check has not been performed yet (NO in step S311), the CPU 21a returns the process to step S301. On the other hand, when the check has been performed for all of the sample IDs registered in the not-validated item list NV (YES in step S311), the CPU 21a ends the list check process.

<Invalid Data Deletion Process>

When many samples are continuously analyzed, many pieces of data are registered in the not-validated item list NV. In general, in a medical facility, an analysis result for which several hours have elapsed since its measurement time will not be validated any more. Therefore, unless data of such an analysis result is deleted from the not-validated item list NV, the data will remain in the not-validated item list NV. The test information management apparatus 2 according to the present embodiment deletes, from the not-validated item list, invalid data for which a predetermined time period has elapsed since its measurement time, by periodically performing an invalid data deletion process described below.

FIG. 15 is a flow chart showing the procedure of the invalid data deletion process performed by the test information management apparatus 2. In the invalid data deletion process, the CPU 21a first obtains the measurement time corresponding to one sample ID from the not-validated item list NV (step S401). Then, the CPU 21a determines whether an elapsed time period since the obtained measurement time exceeds a predetermined reference value (step S402). When the elapsed time period since the measurement time exceeds the reference value (YES in step S402), the CPU 21a creates an e-mail for making notification that sending of analysis results has completed, and sends the created e-mail to the mail address of the handheld device of the subject corresponding to the sample ID (step S403).

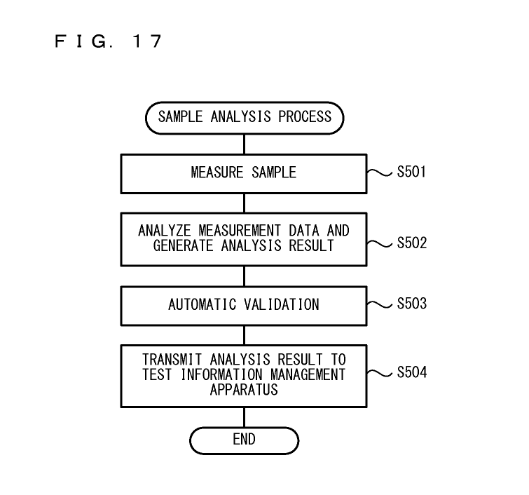

FIG. 16 shows the text of the e-mail sent in step S403. In this case, with respect to the analysis results corresponding to the sample ID, a time period longer than or equal to the reference value has already elapsed since the measurement time. Accordingly, such analysis results will not be validated any more, and data corresponding to the sample ID registered in the not-validated item list NV is considered as invalid. Therefore, text RP4 of the e-mail sent in step S403 includes a message MES3 expressed in character information “Your analysis results have become invalid and will not be sent any more.” The message MES3 allows the subject or the doctor to know that no more analysis result notification e-mail will be sent after this e-mail. Accordingly, the doctor can know that he or she may examine the subject, using the analysis results provided by the e-mails received by the handheld device of the subject up to that point.

When an e-mail for making notification that sending of analysis results has completed as described above has been sent, the CPU 21a deletes data corresponding to the sample ID from the not-validated item list NV (step S404), and advances the process to step S405.

On the other hand, when the elapsed time period since the measurement time does not exceed the reference value (NO in step S402), the CPU 21a advances the process directly to step S405.

In step S405, the CPU 21a determines whether the above-described check has been performed for all of the sample IDs registered in the not-validated item list NV (step S405). When there is a sample ID for which the check has not been performed yet (NO in step S405), the CPU 21a returns the process to step S401. On the other hand, when the check has been performed for all of the sample IDs registered in the not-validated item list NV (YES in step S405), the CPU 21a ends the invalid data deletion process.

### Embodiment 2

### Configuration of Analysis Result Providing System

The configuration of the analysis result providing system according to the present embodiment is similar to that of the analysis result providing system according to Embodiment 1. Therefore, the same components are denoted by the same reference characters and description thereof will be omitted.

[Operation of Analysis Result Providing System]

Next, operations performed by the analysis result providing system 100 according to the present embodiment will be described.

In the analysis result providing system 100 according to the present embodiment, an automatic validation process is performed in each of the sample analyzers 4, 5, and 6.

<Sample Analysis Process>

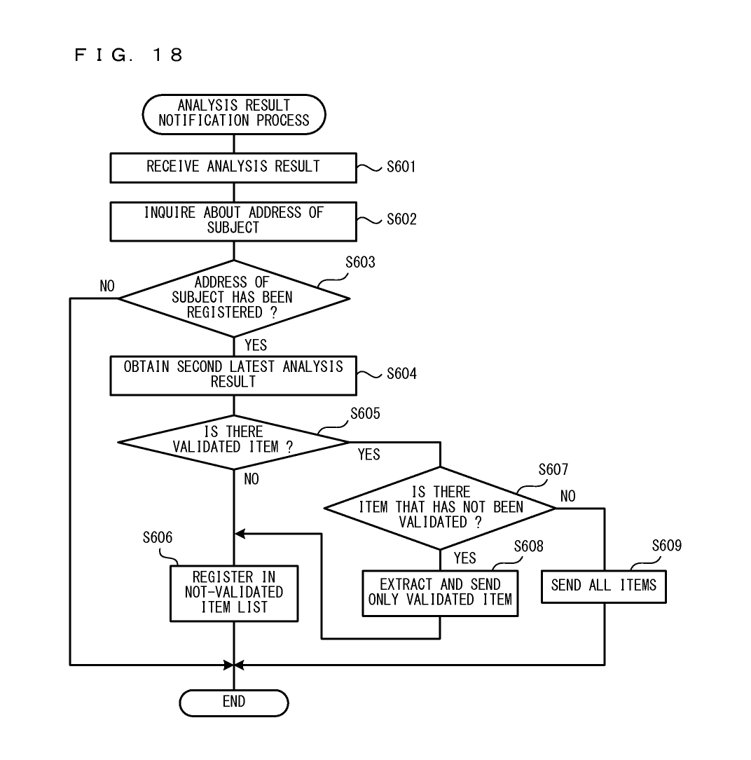

Here, a sample analysis process performed by the sample analyzer 4 will be described. FIG. 17 is a flow chart showing the procedure of the sample analysis process performed by the sample analyzer 4 according to the present embodiment. When sample analysis is to be performed, order information of a subject is transmitted from the test information management apparatus 2 to the sample analyzer 4. The sample analyzer 4 receives the order information, and performs sample measurement for analysis items designated in this order information, by means of the measurement unit 41 (step S501). The measurement data obtained through the sample measurement performed by the measurement unit 41 is provided to the information processing unit 42.

The CPU 421a of the information processing unit 42 analyzes the measurement data and generates analysis results (step S502). Next, the CPU 421a performs an automatic validation process on the analysis results obtained in this manner (step S503). Since this automatic validation process is similar to that described in Embodiment 1, description thereof will be omitted.

Next, the CPU 421a transmits the analysis results for which the automatic validation has been completed, to the test information management apparatus 2 (step S504), and ends the sample analysis process.

<Analysis Result Notification Process>

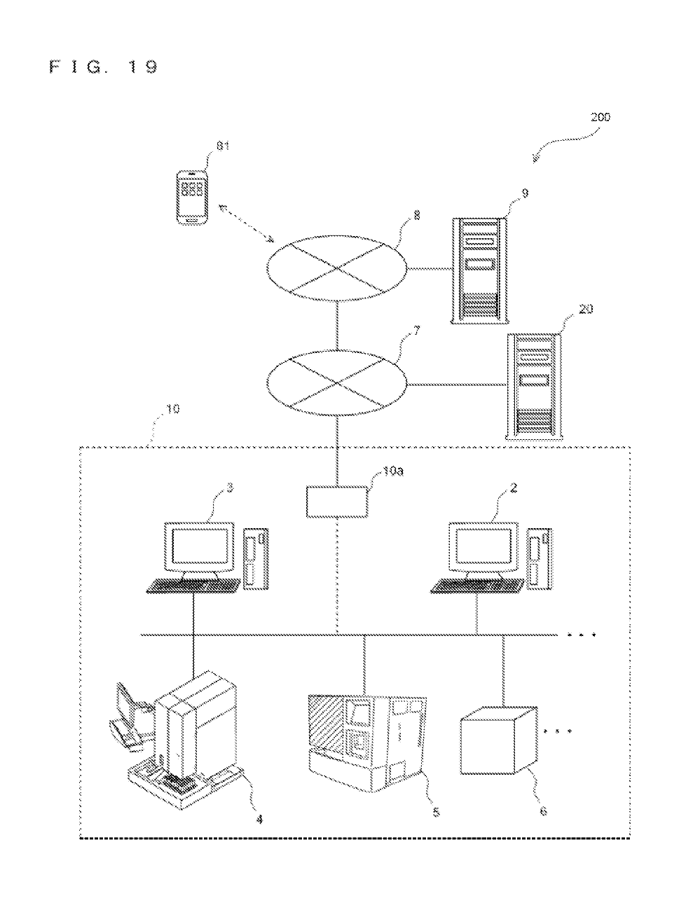

In the present embodiment, when the test information management apparatus 2 has received analysis results transmitted from the sample analyzer 4, 5, or 6, the CPU 21a of the test information management apparatus 2 performs an analysis result notification process described blow. FIG. 18 is a flow chart showing the procedure of the analysis result notification process performed by the test information management apparatus 2 according to the present embodiment. Upon receiving the analysis results (step S601), the CPU 21a of the test information management apparatus 2 requests the subject information management apparatus 3 to inquire about the mail address of the subject, using the subject ID contained in the analysis results as a key (step S602). The subject information management apparatus 3 determines whether the mail address corresponding to the subject ID, that is, the mail address of the handheld device of the subject, has been registered in the subject information database DB3, thereby inquiring about the mail address of the subject. The inquiry result about the mail address of the subject is transmitted from the subject information management apparatus 3 to the test information management apparatus 2.

Upon receiving the inquiry result about the mail address of the subject, the CPU 21a of the test information management apparatus 2 determines whether the mail address of the subject has been registered in the subject information database DB3, based on the received inquiry result (step S603). When the mail address of the subject has not been registered in the subject information database DB3 (NO in step S603), the CPU 21a directly ends the process.

On the other hand, when the mail address of the subject has been registered in the subject information database DB3 (YES in step S603), the CPU 21a obtains, from the database DB2, second latest analysis results of the subject (step S604).

After the process of step S604, the CPU 21a performs the processes of steps S605 to S609 and ends the analysis result notification process. It should be noted that the processes of steps S605 to S609 are similar to those of steps S108 to S112 described in Embodiment 1. Therefore, description thereof will be omitted.

Other operations performed by the analysis result providing system 100 according to the present embodiment are similar to those performed by the analysis result providing system 100 according to Embodiment 1. Therefore, description thereof will be omitted.

According to the above configuration, even when the subject does not send an analysis result notification request mail from the handheld device 81, an analysis result notification e-mail is sent from the test information management apparatus 2 to the handheld device 81 of the subject, and thus, the analysis results are provided to the subject.

### Embodiment 3

### Configuration of Analysis Result Providing System

FIG. 19 is a schematic diagram showing a configuration of an analysis result providing system according to the present embodiment. An analysis result providing system 200 according to the present embodiment includes a web server 20 connected to the Internet 7.

<Configuration of the Web Server 20>

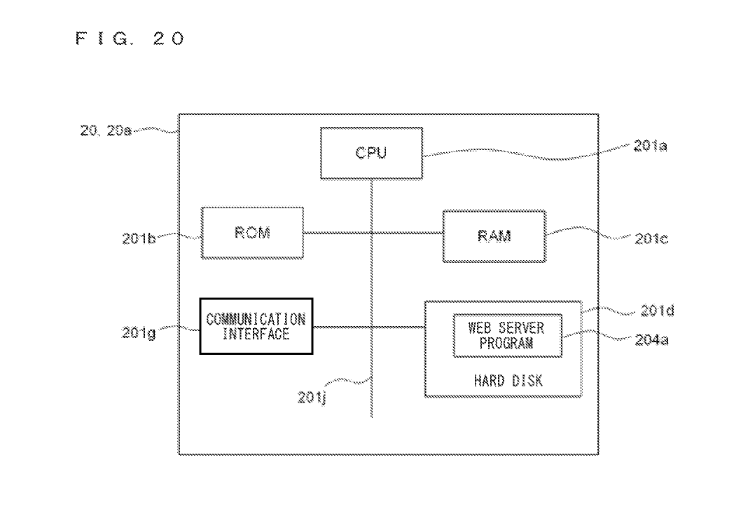

FIG. 20 is a block diagram showing a configuration of the web server 20. The web server 20 is realized by a computer 20a. As shown in FIG. 20, the computer 20a includes a CPU 201a, a ROM 201b, a RAM 201c, a hard disk 201d, and a communication interface 201g. The CPU 201a, the ROM 201b, the RAM 201c, the hard disk 201d, and the communication interface 201g are connected to each other by a bus 201j.

A web server program 204a for causing the computer to function as the web server 20 is installed in the hard disk 201d. By the CPU 201a executing the web server program 204a, the computer 20a functions as the web server 20.

Further, HTML documents are stored in a predetermined area in the hard disk 201d. The presence of each HTML document stored in the hard disk 201d can be specified by an URL (uniform resource locator). When the URL is designated by a web client connected to the Internet 7, the content of the HTML document is displayed on the side of the web client.

Further, the handheld device 81 has a function as a web client. That is, the handheld device 81 can request the web server present in the Internet to transmit an HTML document, through the mobile phone network 8 and the Internet 7, and can receive the HTML document transmitted from the web server and output the content on its display section.

The other configurations of the analysis result providing system according to the present embodiment are similar to those of the analysis result providing system according to Embodiment 1. Therefore, the same components are denoted by the same reference characters and description thereof will be omitted.

[Operation of Analysis Result Providing System]

Next, operations performed by the analysis result providing system 200 according to the present embodiment will be described. In the present embodiment, when the sample analyzer 4, 5, or 6 performs sample analysis, the sample analyzer 4, 5, or 6 transmits analysis results to the test information management apparatus 2, without performing an automatic validation process.

<Analysis Result Notification Process>

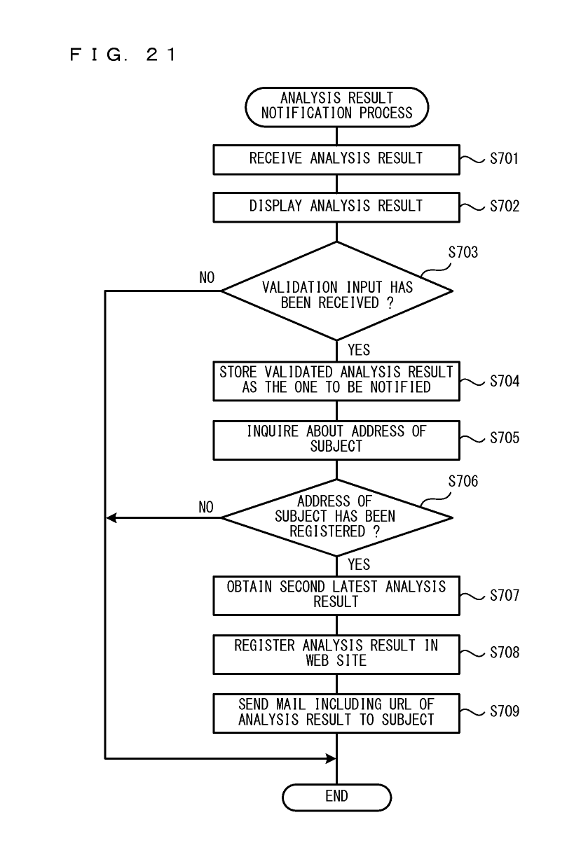

In the present embodiment, when the test information management apparatus 2 has received the analysis results transmitted from the sample analyzer 4, 5, or 6, the CPU 21a of the test information management apparatus 2 performs an analysis result notification process described below. FIG. 21 is a flow chart showing the procedure of the analysis result notification process performed by the test information management apparatus 2 according to the present embodiment. Upon receiving the analysis results (step S701), the CPU 21a of the test information management apparatus 2 causes the display unit 22 to display the analysis results (step S702).

The operator views the displayed analysis results, and performs validation manually. The CPU 21a determines whether one or more validation inputs have been received (step S703). The manual validation is similar to that described in Embodiment 1, and can be performed for each analysis item. For example, when the operator has validated analysis items “WBC” and “RBC”, validation inputs for these items are provided to the CPU 21a, and the validation flags for the items “WBC” and “RBC” are set to ON in their analysis results, respectively.

With respect to an analysis result for each item for which a validation input has been received in step S703, the CPU 21a permits provision of the analysis result to the subject. That is, when one or more validation inputs have been received (YES in step S703), the CPU 21a permits provision of the corresponding one or more analysis results to the subject, and advances the process to step S704. On the other hand, when no validation input has been received (NO in step S703), the CPU 21a does not permit provision of the analysis results to the subject, and ends the analysis result notification process.

When having received one or more validation inputs (YES in step S703), the CPU 21a stores, in the hard disk 21d, the analysis result for each validated analysis item as the one that will be notified of (step S704).

Next, the CPU 21a requests the subject information management apparatus 3 to inquire about the mail address of the subject, using the subject ID contained in the analysis results as a key (step S705). The subject information management apparatus 3 determines whether the mail address corresponding to the subject ID, that is, the mail address of the handheld device of the subject, has been registered in the subject information database DB3, thereby inquiring about the mail address of the subject. The inquiry result about the mail address of the subject is transmitted from the subject information management apparatus 3 to the test information management apparatus 2.

Upon receiving the inquiry result about the mail address of the subject, the CPU 21a of the test information management apparatus 2 determines whether the mail address of the subject has been registered in the subject information database DB3, based on the received inquiry result (step S706). When the mail address of the subject has not been registered in the subject information database DB3 (NO in step S706), the CPU 21a directly ends the process.

On the other hand, when the mail address of the subject has been registered in the subject information database DB3 (YES in step S706), the CPU 21a obtains, from database DB2, second latest analysis results of the subject (step S707).

Next, the CPU 21a creates an HTML document for displaying the analysis result for each validated analysis item and its corresponding second latest value, and uploads the HTML document to the web server 20 (step S708). As a result, the HTML document is stored in the hard disk 201d of the web server 20.

Further, the CPU 21a creates an e-mail in which the URL of the HTML document uploaded to the web server 20 is included in the text, sends the e-mail to the mail address of the handheld device of the subject (step S709), and ends the process.

The handheld device 81 of the subject receives the e-mail sent from the test information management apparatus 2. The text of the e-mail including the URL is displayed on the handheld device 81. By the subject performing an operation of selecting the displayed URL, the handheld device 81 can access the HTML document in the web server 20, which is the link destination. Accordingly, the analysis result(s) and the second latest value(s), which are the contents of the HTML document, are displayed on the display section of the handheld device 81.

Other operations performed by the analysis result providing system 200 according to the present embodiment are similar to those performed by the analysis result providing system 100 according to Embodiment 1. Therefore, description thereof will be omitted.

According to the above configurations, in the sample analyzing systems according to Embodiments 1 and 2, only when provision of analysis results to the subject is permitted, an e-mail is sent to the handheld device of the subject. By receiving the e-mail by means of the handheld device 81, the subject can be provided with the analysis results.

Further, in the sample analyzing system according to Embodiment 3, only when provision of analysis results to the subject is permitted, an e-mail is sent to the handheld device of the subject. By viewing on the handheld device 81 an HTML document specified by the URL designated in the e-mail, the subject can be provided with the analysis results.

Further, in the sample analyzing systems according to Embodiments 1 to 3, when a part of the items of analysis results have been validated but the other items have not been validated, an e-mail for providing the analysis results for the part of the items that have been validated is sent to the mail address of the handheld device of the subject. Accordingly, a situation where none of the analysis results are provided to the subject can be prevented even when provision of analysis results are permitted only for a part of a plurality of items, and analysis results for those permitted items can be provided to the subject. Accordingly, the doctor can use the provided analysis results for that part of the items.

Further, in the sample analyzing systems according to Embodiments 1 to 3, when an analysis result has been validated, provision of the analysis result to the subject is permitted, and when an analysis result has not been validated, provision of the analysis result to the subject is not permitted. Therefore, it is possible to provide with the subject an analysis result that is highly reliable and useful for examination performed by the doctor.

Further, in the sample analyzing systems according to Embodiments 1 to 3, in the case where an analysis result for an item that was not validated once has been later validated, the analysis result for that newly validated item can be provided to the subject. Accordingly, the doctor can use, in examination, analysis results of items previously provided and the newly provided analysis result of that newly validated item.

Further, in the sample analyzing systems according to Embodiments 1 and 2, an analysis result that has satisfied a reliability determination condition in the automatic validation process is validated. Therefore, the validated analysis result is highly reliable, and can be useful in the examination performed by the doctor.

Further, in the sample analyzing systems according to Embodiments 1 to 3, it is possible to provide the subject with a highly reliable analysis result, which has been validated by an operator, through the manual validation process. Therefore, the doctor can effectively use the analysis result in the examination of the subject.

### Other Embodiments

In Embodiments 1 to 3 described above, configurations have been described in which the test information management apparatus 2 determines whether to permit provision of analysis results to the subject, and sends an e-mail for providing the analysis results that have been permitted to be provided to the subject, to the handheld device 81 of the subject. However, the present invention is not limited thereto. Each sample analyzer may determine whether to permit provision of analysis results to the subject, and send an e-mail for providing the analysis results that have been permitted to be provided to the subject, to the handheld device 81 of subject.

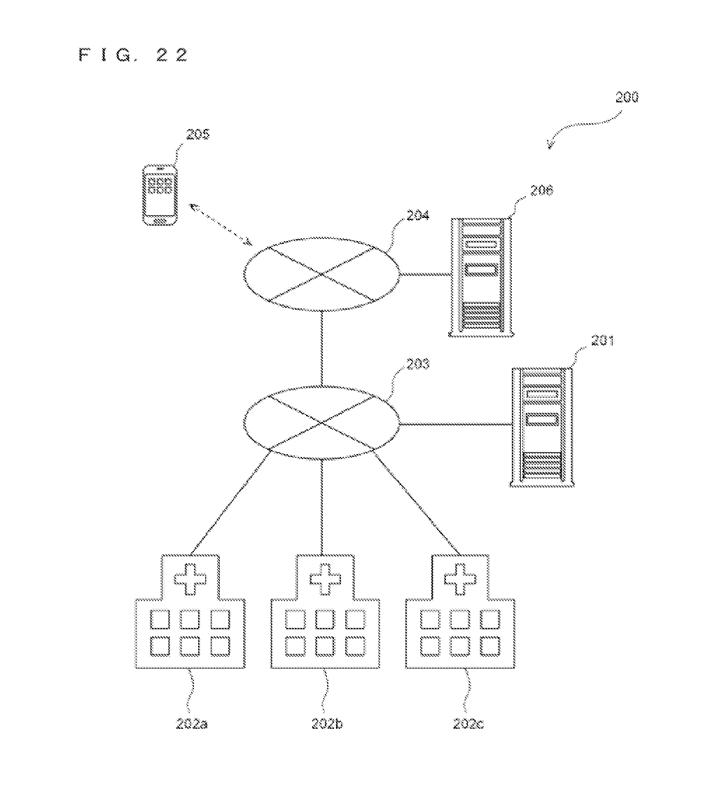

Further, for example, a configuration shown in FIG. 22 may be employed. FIG. 22 is a schematic diagram showing a configuration of an analysis result providing system according to another embodiment. The analysis result providing system 200 includes a test information management server 201, a handheld device 205, and a mail server 206. The mail server 206 is connected to a mobile phone network 204, and the handheld device 205 is communicable with another handheld device, the mail server 206, and the test information management server 201, via the mobile phone network 204. The test information management server 201 is connected to Internet 203. Further, a plurality of medical facilities 202a, 202b, and 202c are connected to the Internet 203. Sample analyzers are installed in each of the medical facilities 202a, 202b, and 202c. Each sample analyzer installed in each of the medical facilities 202a, 202b, and 202c can perform data communication with the test information management server 201. The test information management server 201 has functions as the test information management apparatus 2, the subject information management apparatus 3, an e-mail client, and a web server. The test information management server 201 is a cloud server for the medical facilities 202a, 202b, and 202c, manages order information for the sample analyzers installed in the medical facilities 202a, 202b, and 202c, and manages analysis results obtained by the sample analyzers installed in the medical facilities 202a, 202b, and 202c. In this configuration, in the case where a sample of a subject is analyzed in the medical facility 202a, 202b, or 202c, and analysis results are validated, the test information management server 201 permits provision of the validated analysis results to the subject, creates an HTML document for viewing the validated analysis results, and stores it in a storage area in the test information management server 201. Further, the test information management server 201 creates an e-mail including the URL of the HTML document, and sends the e-mail to the mail address of the handheld device 205 of the subject. Accordingly, it is possible for the plurality of medical facilities 202a, 202b, and 202c to easily introduce the system of providing analysis results to the subject.

In Embodiments 1 to 3 described above, configurations have been described in which the test information management apparatus 2 and the subject information management apparatus 3 are provided separately. However, the present invention is not limited thereto. One apparatus having the function of the test information management apparatus 2 and the function of the subject information management apparatus 3 may be used.

Further, in Embodiments 1 to 3 described above, configurations have been described in which a single computer 2a executes all of the processes of the computer program 24a. However, the present invention is not limited thereto. The processes similar to those of the computer program 24a described above may be executed by a plurality of apparatuses (computers) in a distributed system. The same applies to the subject information management apparatus 3 and the information processing unit 42.

The analysis result providing systems, the sample analyzing systems, the analysis result providing apparatuses, the sample analysis result providing methods and the computer programs according to the present invention are useful as analysis result providing systems for providing results of analysis of a sample such as blood or urine collected from a subject such as a patient, to the subject or the like; sample analyzing systems for analyzing samples; analysis result providing apparatuses, sample analysis result providing methods; and computer programs; and the like.

What is claimed is:

1. An analysis result providing system comprising:
a sample analyzing section which analyzes a sample collected from a subject;
a validation section which validates an analysis result obtained by the sample analyzing section;
an e-mail sending section which sends, when an analysis result of the sample collected from the subject has been validated by the validation section, an e-mail for providing the analysis result; and
a handheld device which is carried by the subject and which is capable of receiving the e-mail sent by the e-mail sending section and displaying a content of the e-mail.

2. A sample analyzing system comprising:
a sample analyzing section which analyzes a sample collected from a subject;
a validation section which validates an analysis result obtained by the sample analyzing section; and
an e-mail sending section which sends, when an analysis result of the sample collected from the subject has been validated by the validation section, an e-mail for providing the analysis result to a handheld device carried by the subject.

3. An analysis result providing apparatus, comprising:
an analysis result obtaining section which obtains an analysis result of a sample collected from a subject;
a validation section which validates an analysis result obtained by the analysis result obtaining section; and
an e-mail sending section which sends, when an analysis result of the sample collected from the subject has been validated by the validation section, an e-mail for providing the analysis result to a handheld device carried by the subject.

4. The analysis result providing apparatus of claim 3, further comprising:
an e-mail receiving section which receives an analysis result requesting e-mail sent from the handheld device carried by the subject, wherein
the e-mail sending section is configured to send the e-mail for providing the analysis result to the handheld device when the analysis result requesting e-mail has been received by the e-mail receiving section.

5. The analysis result providing apparatus of claim 4, wherein
the validation section is configured to validate, when the analysis result requesting e-mail has been received by the e-mail receiving section, the analysis result of the sample collected from the subject who sent the e-mail.

6. The analysis result providing apparatus of claim 3, wherein
the analysis result obtaining section is configured to obtain analysis results of a sample collected from a subject, the analysis results respectively obtained by a plurality of sample analyzers, and
the e-mail sending section is configured to send to the handheld device the e-mail for providing each of the analysis results obtained by the plurality of sample analyzers when each of the analysis results obtained by the plurality of sample analyzers has been validated by the validation section.

7. The analysis result providing apparatus of claim 3, wherein
the analysis result obtaining section is capable of obtaining a sample analysis result that includes respective analysis results for a plurality of items,
the validation section is capable of validating the analysis results obtained by the analysis result obtaining section for the respective items, and
the e-mail sending section is configured to send, when analysis results for only a part of the items have been validated by the validation section, an e-mail for providing the analysis results for the part of the items to the handheld device.

8. The analysis result providing apparatus of claim 7, wherein
the e-mail sending section is configured to send, when analysis results for only a part of the items have been validated by the validation section, the e-mail for providing the analysis results for only the part of the items.

9. The analysis result providing apparatus of claim 8, further comprising:
a second validation section which further validates, when analysis results for a part of the items have been validated by the validation section and an analysis result for another item has not been validated, the analysis result for the another item, wherein
the e-mail sending section is configured to send, when the analysis result for the another item has been validated by the second validation section, a second e-mail for providing the analysis result for the another item, and is configured not to send, when the analysis result for the another item has not been validated by the second validation section, the second e-mail for providing the analysis result for the another item.

10. The analysis result providing apparatus of claim 9, wherein
the e-mail sending section is configured to send the second e-mail for providing the analysis results for all of the part of the items that have been validated by the validation section and the another item that has been validated by the second validation section.

11. The analysis result providing apparatus of claim 7, wherein
the validation section is configured to determine, for the respective items, whether the analysis results obtained by the analysis result obtaining section satisfy their corresponding predetermined reliability determination conditions, and is configured to validate an analysis result for an item that satisfies its corresponding reliability determination condition and not to validate an analysis result for an item that does not satisfy its corresponding reliability determination condition.

12. The analysis result providing apparatus of claim 9, wherein
the second validation section is configured to receive from an operator a validation instruction for validating an analysis result on an item-by-item basis, and is configured to validate an analysis result for an item for which the validation instruction has been received from the operator and not to validate an analysis result for an item for which the validation instruction has not been received from the operator.

13. The analysis result providing apparatus of claim 3, wherein
the validation section is configured to determine whether the analysis result obtained by the analysis result obtaining section satisfies a predetermined reliability determination condition, and is configured to validate the analysis result when the analysis result satisfies the reliability determination condition and not to validate the analysis result when the analysis result does not satisfy the reliability determination condition.

14. The analysis result providing apparatus of claim 3, wherein
the validation section is configured to receive from an operator a validation instruction for validating an analysis result, and is configured to validate an analysis result for which the validation instruction has been received from the operator and not to validate an analysis result for which the validation instruction has not been received from the operator.

15. The analysis result providing apparatus of claim 3, wherein
the e-mail sending section is configured to send, when an analysis result has been validated by the validation section, the e-mail including the analysis result of the sample collected from the subject, to the handheld device.

16. The analysis result providing apparatus of claim 15, wherein
the e-mail sending section is configured to send, when an analysis result has been validated by the validation section, the e-mail further including analysis result normal range information, to the handheld device.

17. The analysis result providing apparatus of claim 15, wherein
the e-mail sending section is configured to send, when an analysis result has been validated by the validation section, the e-mail further including analysis result describing information, to the handheld device.

18. The analysis result providing apparatus of claim 3, further comprising:
a storage section which stores, when an analysis result has been validated by the validation section, the analysis result that has been validated in a storage area accessible by the handheld device, wherein
the e-mail sending section is configured to send, to the handheld device, the e-mail including information for accessing the storage area in which the analysis result has been stored by the storage section.

19. A method of providing a sample analysis result, comprising:
an analyzing step of analyzing a sample collected from a subject;
a validation step of validating an analysis result obtained in the analyzing step;
a sending step of sending, when an analysis result of the sample collected from the subject has been validated in the validation step, an e-mail for providing the analysis result to a handheld device carried by the subject; and
a receiving step of the subject receiving the e-mail by means of the handheld device.
